(19) United States
(12) Patent Application Publication
(10) Pub. No.: US 2010/0094419 A1
(43) Pub. Date: Apr. 15, 2010

(54) Porous composite comprising silicon-substituted hydroxyapatite and beta-tricalcium phosphate, and process for preparing the same

(75) Inventors: Min Sung Kim, Chungcheongbuk-do (KR); Seong Min Ha, Chungcheongbuk-do (KR); Young Muk Choi, Chungcheongbuk-do (KR)

Correspondence Address: SHERIDAN ROSS PC, 1560 BROADWAY, SUITE 1200, DENVER, CO, 80202, US

(73) Assignee: METABIOMED.CO.LTD, Chungcheongbuk-do (KR)

(21) Appl. No.: 12/456,876

(22) Filed: Jun. 24, 2009

(30) Foreign Application Priority Data
Oct. 9, 2008 (KR) 1020080098993

(51) Int. Cl. A61F 2/28; C04B 35/00

(52) U.S. Cl. 623/16.11; 106/691

(57) ABSTRACT

There are disclosed a porous composite comprising silicon-substituted hydroxyapatite and β-tricalcium phosphate (β-TCP), and a method for preparing the same. The porous composite is prepared by subjecting natural coral to hydrothermal and solvothermal reactions to prepare silicon-substituted hydroxyapatite (Si-HA) and subjecting the Si-HA to a heat treatment process. Thereafter, Si-HA and β-TCP may be mixed in the porous composite. As a result, the porous composite is excellent in biocompatibility and biodegradability. Also, the porous composite functions to maintain the microstructure of coral and is similar to natural bone in terms of composition and shape. Accordingly, the porous composite may be effectively used as a bone tissue repairing material and a bone graft material that can substitute for human hard tissue.

### BACKGROUND OF THE INVENTION

1. Field of the Invention

The present invention relates, in general, to a porous composite comprising silicon-substituted hydroxyapatite and β-tricalcium phosphate (β-TCP) and a method for preparing the same.

2. Description of the Related Art

Calcium phosphate-based materials are widely used as bone tissue-repairing materials and bone graft materials. Hydroxyapatite (HA) is the most widely used among these.

Hydroxyapatite has characteristics very similar to those of the hard tissue of human bones, teeth and the like in crystallographic and chemical aspects. Therefore, when hydroxyapatite is grafted into living tissues in vivo, no harmful reaction occurs with the living tissues, and it is spontaneously engrafted into peripheral tissues. In fact, hydroxyapatite is composed of more than 95% enamel, and bone is a complex of fibrous protein (i.e., collagen) and approximately 65% hydroxyapatite. Owing to its excellent biocompatibility and bioactivity, hydroxyapatite has been increasingly considered as a material that can be used to substitute for damaged teeth or bones. However, hydroxyapatite is not suitable as a material for those types of living hard tissue which require high mechanical strength or resistance to fracturing, such as artificial teeth or hip joints, since the hydroxyapatite has poor mechanical properties, such as mechanical strength and fracture toughness. However, hydroxyapatite is restrictively used for organs that do not require high mechanical strength, such as ear ossicles. Also, hydroxyapatite has a problem in that it is difficult to use it as a substitute for autogenous bone due to the very low in vivo absorbency of the hydroxyapatite.

In order to improve the low mechanical strength of hydroxyapatite, compounding hydroxyapatite with other materials has been attempted. That is, hydroxyapatite was mixed with metals or ceramics having excellent mechanical properties to improve the mechanical properties of hydroxyapatite, and to make use of the biocompatibility and bioactivity of hydroxyapatite as well. However, in the process used for preparing a complex of hydroxyapatite and metals or ceramics, apatite may become dehydrated and degraded during heat treatment due to the contact of the hydroxyapatite with the metal or ceramics.

In order to improve the low in vivo absorbency of hydroxyapatite and enhance its biodegradability, β-tricalcium phosphate (β-TCP), taken as an example of calcium phosphate-based compounds, has been used. However, β-TCP has a problem in that the strength of β-TCP is not maintained for the period of its degradation since it has low mechanical strength.

As described above, much research has been conducted into how to improve the low mechanical strength and low in vivo absorbency of hydroxyapatite. In particular, in order to make use of both the biocompatibility and bioactivity of hydroxyapatite and the biodegradability of β-TCP, much research has been conducted with the goal of finding a method for preparing hydroxyapatite in the form of biphasic calcium phosphate by incorporating β-TCP into the structure of hydroxyapatite. Conventional and simple mixing methods have been used to prepare hydroxyapatite in the form of biphasic calcium phosphate, but these mixing methods have disadvantages in that the manufacturing process is ineffective and the mixing ratio may be varied according to each different manufacturing process.

Meanwhile, a small amount of other ions are used to substitute for the Ca, P and O ions in the structure of the apatite that structurally constitutes bone, and thus they act as important factors that affect the surface charge, surface structure, strength, and solubility of hydroxyapatite. In addition to apatite, bioactive ceramics that have been widely used as natural bone substitutes contain a large amount of silicon and magnesium ions. According to a theory proposed by Kokubo, et al., it was reported that silicon is slowly eluted from glass ceramics in simulated body fluid, and is present in a surface of the glass ceramics in the form of silicate ions. Here, the silicate ions function to induce new apatite nuclei formation to rapidly form an apatite layer on the surface of the glass ceramics. Also, Carlise, et al. emphasized the importance of silicon in bone formation using electron microscopic studies. Also, it was known that Si ions function to enhance the important characteristics (i.e. mechanical strength) of porous ceramics, and to promote the bioactivity of apatite as well.

Therefore, there has been much research into methods which promote the mechanical strength and bioactivity by substituting Si ions into hydroxyapatite. For example, a porous complex of Si-containing calcium phosphate-based complex compounds and a method for preparing the same using natural coral have been reported, wherein the natural coral has the structure similar to that of human trabecular bone since it is composed of calcium carbonate and its pores have a diameter of 200 μm to 500 μm and are connected with each other in three dimensions. More specifically, a porous complex of Si-containing calcium phosphate-based complex compounds and a method for preparing the same have been reported, comprising converting natural coral into apatite through a hydrothermal reaction while maintaining the microstructure of the natural coral [see U.S. Pat. Nos. 3,890,107 and 3,929,971, and Biomaterials 17(17), p1709, 1996, Material Characterization 47(2), p83, 2001], and subjecting the apatite to solvothermal reaction [see Korean Registered Patent No. 10-475828 and U.S. Pat. No. 7,008,450]. However, the porous complex of Si-containing calcium phosphate-based complex compounds has a disadvantage in that it does not enhance bioactivity since it is impossible to improve the biodegradability of the porous complex.

Ideal biodegradable material should satisfy requirements including excellent biocompatibility, maintenance of strength and stability after bone grafting, and gradual degradation and substitution with new bone over time.

Accordingly, there is an urgent demand for development of bone tissue repairing materials and bone graft materials that have excellent mechanical strength and which may substitute for human hard tissue by the activation of their biocompatibility and biodegradability.

The present inventors have carried out studies to develop bone tissue repairing materials and bone graft materials that have excellent mechanical strength and which may substitute for human hard tissue by the activation of their biocompatibility and biodegradability, and found that a porous composite may be obtained by preparing silicon-substituted hydroxyapatite by subjecting natural coral to hydrothermal and solvothermal reactions and converting a portion of the silicon-substituted hydroxyapatite into β-TCP through a heat treatment process. Also, the present inventors have found that, when the porous composite is grafted into the region of a bone defect, new bone begins to be formed from the edge of the bone defect region (new bone formation). As a result and based on the above-mentioned facts, the present invention was completed.

### SUMMARY OF THE INVENTION

Accordingly, the present invention has been made keeping in mind the above problems occurring in the prior art, and an object of the present invention is to provide a porous composite comprising silicon-substituted hydroxyapatite and β-tricalcium phosphate (β-TCP) capable of improving the biocompatibility of coral with maintenance of its own inherent structure and of being gradually absorbed in vivo to substitute for new bone.

Another object of the present invention is to provide a method for preparing the same.

In order to accomplish the above objects, the present invention provides a porous composite comprising silicon-substituted hydroxyapatite and β-tricalcium phosphate (β-TCP).

In this case, the porous composite may be composed of 50 to 90% by weight of silicon-substituted hydroxyapatite and 10 to 50% weight of β-TCP.

Also, content of the substituted silicon in the porous composite may be in a range of 0.1 to 2.0% by weight, based on the total weight of the porous composite.

Also, the present invention provides a method for preparing the porous composite. Here, the method includes: 1) preparing silicon-substituted hydroxyapatite by subjecting natural coral to hydrothermal reaction in a (NH₄)₂HPO₄ solution, followed by subjecting the hydrothermally reacted coral to solvothermal reaction in a silicon acetate/acetone-saturated solution; and 2) converting a portion of the silicon-substituted hydroxyapatite into β-tricalcium phosphate (β-TCP) by performing a heat treatment process on the silicon-substituted hydroxyapatite prepared in Step 1.

In this case, the hydrothermal reaction of Step 1 may be performed at 200° C. for 16 to 20 hours.

Also, the solvothermal reaction of Step 1 may be performed at 50 to 90° C. for 30 to 50 hours.

In addition, the heat treatment process of Step 2 may be performed at 800 to 1200° C. for 1 to 6 hours.

Additionally, the present invention provides a bone tissue repairing material comprising the porous composite according to one exemplary embodiment of the present invention.

Furthermore, the present invention provides a bone graft material comprising the porous composite according to one exemplary embodiment of the present invention.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a photograph showing a porous composite comprising silicon-substituted hydroxyapatite and β-TCP according to one exemplary embodiment of the present invention;

FIG. 2 is a diagram showing the X-ray diffraction (XRD) analysis of the porous composite comprising silicon-substituted hydroxyapatite and β-TCP according to one exemplary embodiment of the present invention, taken using an X-ray diffractometer;

FIG. 3A is a diagram showing the XRD analysis of natural coral, and FIG. 3B is a diagram showing the XRD analysis of silicon-substituted hydroxyapatite;

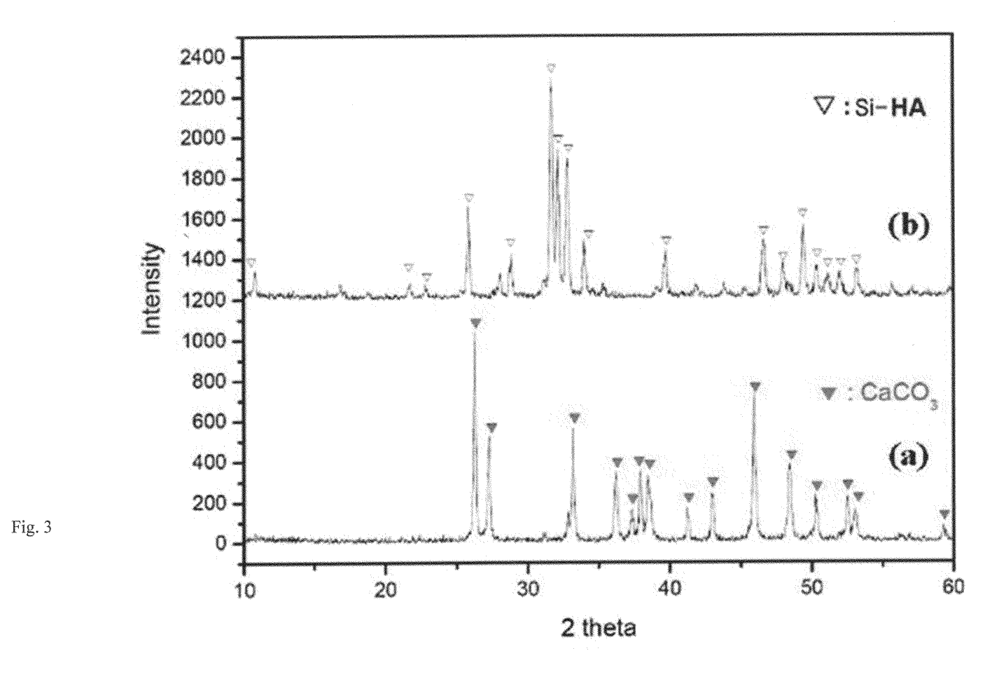

FIG. 4 is a diagram showing tissue 4 weeks after the grafting of the porous composite according to one exemplary embodiment of the present invention into the region of a bone defect, as photographed using an X-ray imaging system;

FIG. 5 is a diagram showing tissue 8 weeks after the grafting of the porous composite according to one exemplary embodiment of the present invention into a defective bone region, as photographed under an X-ray imaging system;

FIG. 6 is a diagram showing tissue 4 weeks after the grafting of the porous composite according to one exemplary embodiment of the present invention into a bone defect region, as photographed under a microscope [(A) Negative control (H&E staining), (B) Negative control (Masson's Trichrome staining), (C) Positive control (H&E staining), (D) Comparative group (Masson's Trichrome staining), (E) Experimental group A (Masson's Trichrome staining), (F) Experimental group B (Masson's Trichrome staining), and (G) Experimental group C (Masson's Trichrome staining)];

FIG. 7 is a diagram showing tissue 8 weeks after the grafting of the porous composite according to one exemplary embodiment of the present invention into the region of a bone defect, as photographed under a microscope [(A) Negative control, (B) Positive control, (C) Comparative group, (D) Experimental group A, (E) Experimental group B, and (F) Experimental group C: All the experimental groups are stained in the Masson's Trichrome staining method]; and

FIG. 8 is a diagram showing the new bone mineral density following the grafting of the porous composite according to one exemplary embodiment of the present invention into the region of a bone defect, as measured with an image analyzer.

### DESCRIPTION OF THE PREFERRED EMBODIMENTS

Hereinafter, exemplary embodiments of the present invention will be described in more detail.

The porous composite according to one exemplary embodiment of the present invention is composed of 50 to 90% by weight of silicon-substituted hydroxyapatite (Si-HA) and 10 to 50% weight of β-TCP, and preferably composed of 60% by weight of Si-HA and 40% weight of β-TCP.

The substituted silicon in the porous composite according to one exemplary embodiment of the present invention has a content of 0.1 to 2.0% by weight, and preferably 0.5 to 1.0% by weight, based on the total weight of the porous composite. When the content of the substituted silicon in the porous composite is within the range, the porous composite shows excellent biocompatibility.

Also, the method for preparing a porous composite according to one exemplary embodiment of the present invention includes: preparing silicon-substituted hydroxyapatite by subjecting natural coral to hydrothermal reaction in a (NH₄)₂HPO₄ solution, followed by subjecting the hydrothermally reacted coral to solvothermal reaction in a silicon acetate/acetone-saturated solution; and converting a portion of the silicon-substituted hydroxyapatite into β-tricalcium phosphate (β-TCP) by performing a heat treatment process on the silicon-substituted hydroxyapatite.

The hydrothermal reaction is preferably performed at 200° C. for 16 to 20 hours, and the solvothermal reaction is preferably performed at 50 to 90° C. for 30 to 50 hours.

The heat treatment process is preferably performed at 800 to 1200° C. for 1 to 6 hours, and preferably for 1 to 3 hours. After the heat treatment process, the silicon-substituted hydroxyapatite and β-TCP may be mixed together in the porous composite. As a result, the porous composite has excellent biocompatibility and biodegradability.

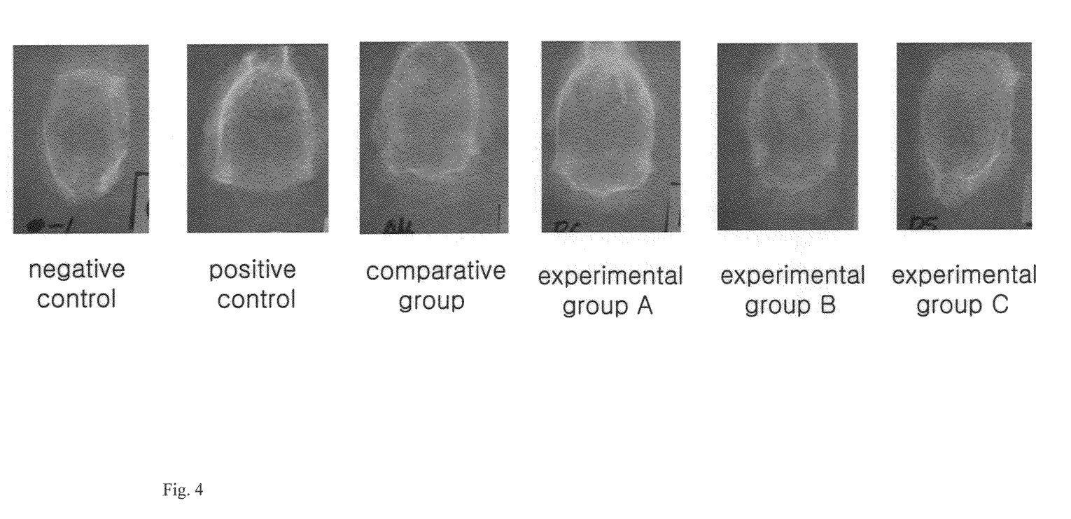

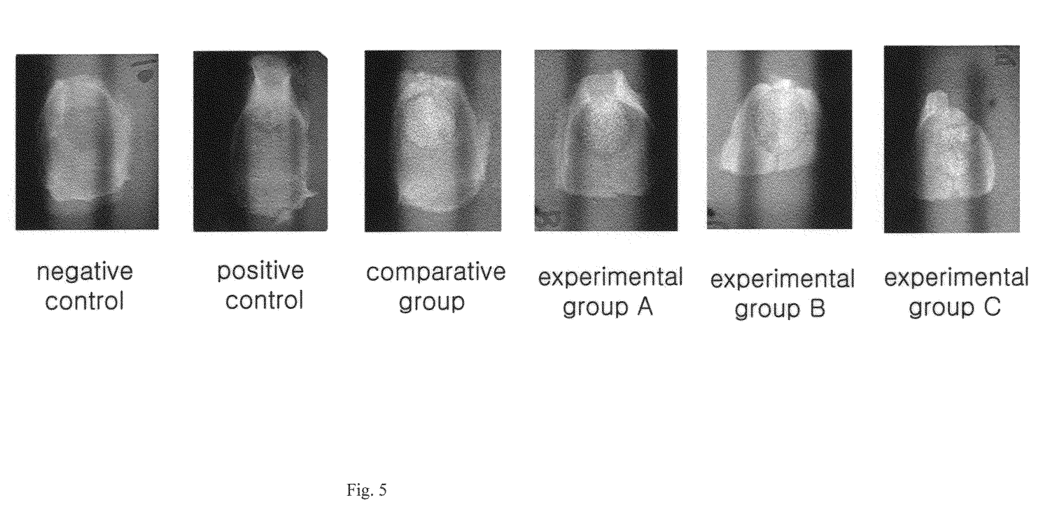

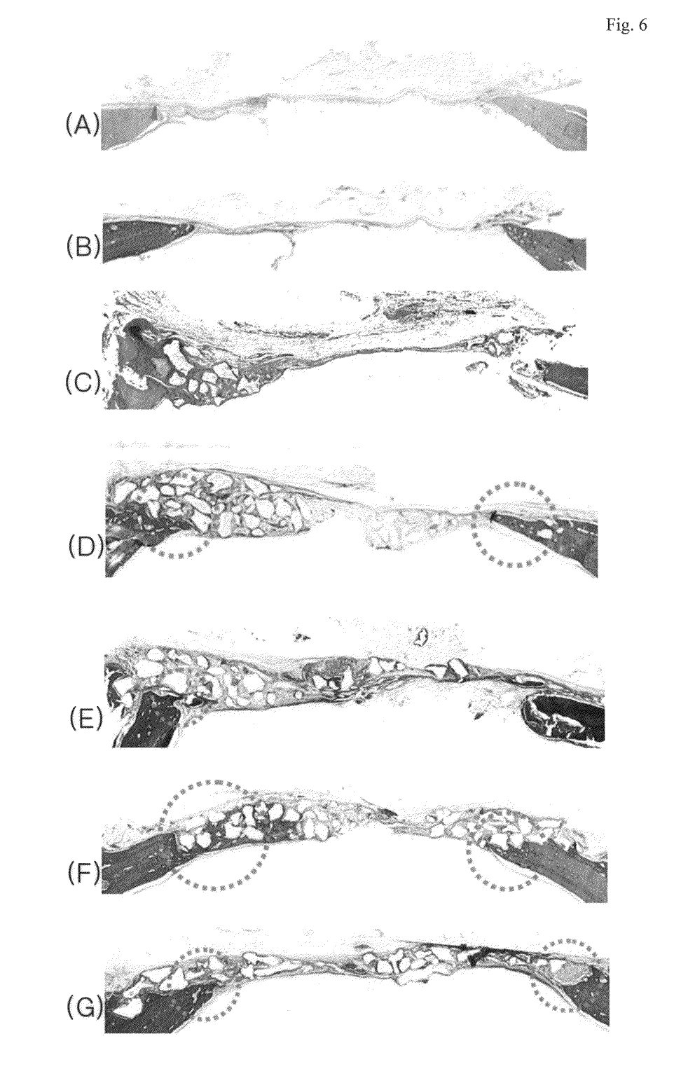

The porous composite prepared in the method according to one exemplary embodiment of the present invention is grafted into each defective bone region, and tissues of the defective bone regions are photographed under an X-ray imaging system after the 4^{th} and 8^{th} graftings of the porous composite. As a result, it was revealed that the porous composite grafted into each defective bone region stays fixed there (see FIGS. 4 and 5). From the microscopic results, it is revealed that new bone successfully forms from the edge of the defective bone region (new bone formation) (see FIGS. 6 to 8).

The porous composite prepared in the method according to one exemplary embodiment of the present invention has a tetrahedral structure. This tetrahedral structure of the porous composite indicates that silicon is substituted in a position P of a hydroxyapatite structure, and is present in the form of a silicate. Also, the porous composite functions to maintain the microstructure of coral and is similar to natural bones in terms of composition and shape. Accordingly, the porous composite according to one exemplary embodiment of the present invention may be effectively used as a bone tissue repairing material and a bone graft material which can substitute for human hard tissue.

A better understanding of the present invention may be obtained through the following examples which are set forth to illustrate, but are not to be construed as limiting the present invention.

### EXAMPLES

### Example 1

### Preparation of a Porous Composite Comprising Silicon-Substituted Hydroxyapatite and β-Tricalcium Phosphate (β-TCP)

Natural coral having an aragonite crystal phase of CaCO₃ as the main component was immersed in 30% sodium hypochlorite (NaOCl) solution and water for 48 hours, and organic matter and impurities were removed from the natural coral using an ultrasonic cleaner.

500 g of coral and 5 l of 2M (NH₄)₂HPO₄ solution were put into the container (10 l) of a Teflon-coated hydrothermal synthesizer, and subjected to hydrothermal reaction at 200° C. for 20 hours. Then, 500 g of the hydrothermally reacted coral and 5 l of a silicon acetate/acetone saturated solution were put into a container (10 l) of a Teflon-coated hydrothermal synthesizer, and the container was sealed. Thereafter, the resulting mixture was subjected to solvothermal reaction at 70° C. for 40 hours to prepare a silicon-substituted hydroxyapatite porous complex. The prepared silicon-substituted hydroxyapatite porous complex was washed with acetone and distilled water in an ultrasonic cleaner, dried, and then heated respectively at 800° C., 1000° C. and 1200° C. for 1 to 3 hours in an electric sintering furnace. Samples of the heated, prepared silicon-substituted hydroxyapatite porous complex were then spontaneously cooled to prepare porous composites.

### Example 2

### Preparation of a Porous Composite Comprising Silicon-Substituted Hydroxyapatite and β-TCP

Porous composites were prepared in the same manner as in Example 1, except that the hydrothermal reaction in Example 1 was performed for 16 hours instead of 20 hours.

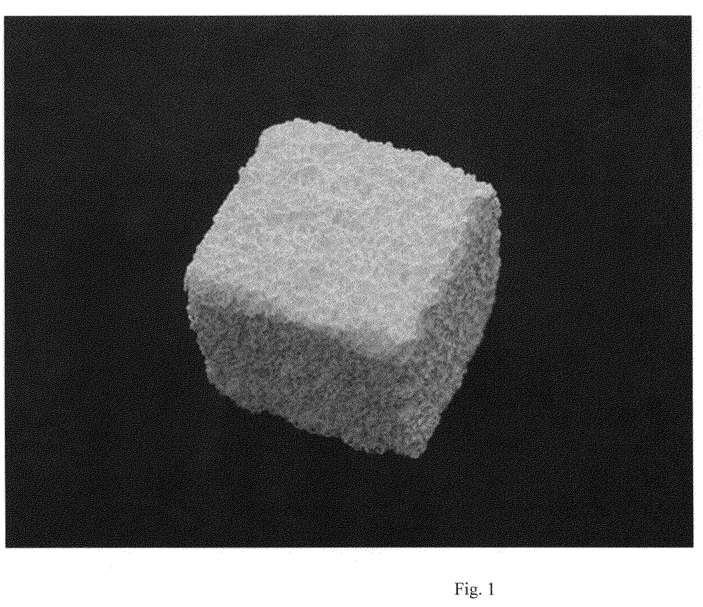

A camera was used to photograph the prepared porous composites. The photographed images of the porous composites are shown in FIG. 1.

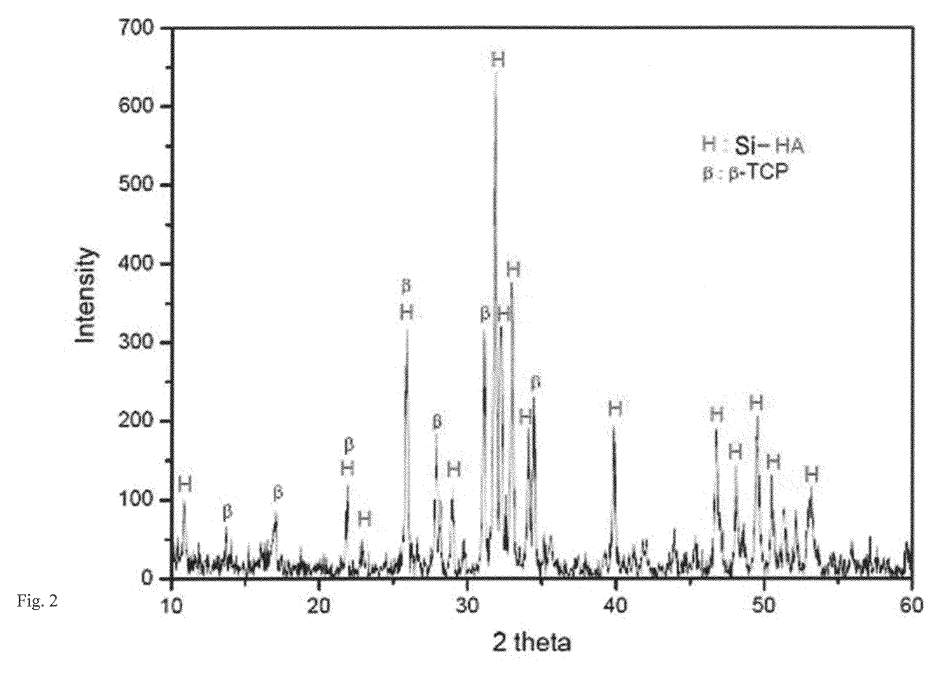

XRD data was obtained from the prepared porous composites using a MacScience diffractometer that uses Cu Kα radiation, and the XRD analyses of the porous composites are shown in FIG. 2.

A phase fraction of the porous composite was calculated from the XRD data. The results are listed in the following Table 1. Also, an inductively coupled plasma spectrometer (ICP) was used to calculate a content of the substituted silicon. The results are listed in the following Table 2.

TABLE 1
Phase fractions of porous composites

| | Heat | | | | |
|---|---|---|---|---|---|
| | treatment | Final | | | |
| | time | phase | 800° C. | 1000° C. | 1200° C. |
| Ex. 1 | 1 hr | Si-HA | 69.1% | 73.6% | 77.8% |
| [Hydrothermal | | β-TCP | 30.9% | 26.4% | 22.2% |
| Rxn at 200° C., | 2 hrs | Si-HA | 70.4% | 74.3% | 78.5% |
| 20 hrs] | | β-TCP | 29.6% | 25.7% | 21.5% |
| | 3 hrs | Si-HA | 71.5% | 75.9% | 80.4% |
| | | β-TCP | 28.5% | 24.1% | 19.6% |
| Ex. 2 | 1 hr | Si-HA | 50.1% | 54.9% | 58.6% |
| [Hydrothermal | | β-TCP | 49.9% | 45.1% | 41.4% |
| Rxn at 200° C., | 2 hrs | Si-HA | 52.8% | 55.8% | 59.2% |
| 16 hrs] | | β-TCP | 47.2% | 44.2% | 40.8% |
| | 3 hrs | Si-HA | 53.4% | 56.2% | 58.9% |
| | | β-TCP | 46.6% | 43.8% | 41.1% |

TABLE 2
Contents of substituted silicon in porous composites

| | Heat treatment | Si Content (wt %) | | |
|---|---|---|---|---|
| | Time | 800° C. | 1000° C. | 1200° C. |
| Ex. 1 | 1 hr | 0.67 | 0.65 | 0.68 |
| [Hydrothermal Rxn | 2 hrs | 0.64 | 0.65 | 0.64 |
| at 200° C., 20 hrs] | 3 hrs | 0.66 | 0.67 | 0.64 |
| Ex. 2 | 1 hr | 0.74 | 0.72 | 0.78 |
| [Hydrothermal Rxn | 2 hrs | 0.71 | 0.74 | 0.74 |
| at 200° C., 16 hrs] | 3 hrs | 0.75 | 0.70 | 0.75 |

As shown in FIG. 1, it was seen that the porous composite according to the present invention has a tetrahedral structure, and thus silicon is present in the form of silicate since the silicon is substituted in the position occupied by P in the hydroxyapatite structure.

As shown in FIG. 2, it was also confirmed that the porous composite according to the present invention includes a mixture of silicon-substituted hydroxyapatite and β-TCP.

Also as listed in Table 1, it was confirmed that the phase fraction of the porous composite according to the present invention is formed so that a weight ratio of Si-HA to β-TCP can be 50 to 90%: 10 to 50%.

As listed in Table 2, it was also confirmed that the content of the substituted silicon in the porous composite according to the present invention is in a range of 0.1 to 2.0% by weight, and preferably 0.5 to 1.0% by weight, based on the total weight of the porous composite.

### Comparative Example 1

### Preparation of a Silicon-Substituted Hydroxyapatite Porous Complex

A silicon-substituted hydroxyapatite porous complex was prepared by performing just the hydrothermal and solvothermal reactions as described in Example 1.

### Experimental Example 1

### Bone Regeneration Test and Bone Grafting

In order to determine whether the porous composite according to the present invention could substitute for human hard tissue, bone regeneration tests and bone grafting were performed as follows.

1. Bone Graft Targets and Experimental Groups

60 male Sprague Dawley (SD) rats weighing 250 to 300 g were divided into two groups: a 4-week grafted group (30 rats) and an 8-week grafted group (30 rats). For each of the 4-week and 8-week grafted groups, 3 out of 30 rats in each group were allotted to the negative control (None) and the positive control (MBCP bone substitute: HA:β-TCP=60%:40%, Biomatlante), respectively, and 6 rats were allotted to the comparative group, the experimental groups A, B and C, respectively. The grafted materials are listed in the following Table 3. One laboratory animal was bred per cage during the experimental period, and hard food (PicoLab Rodnet Diet 20 (Nutrition™ 20% protein diet formulated for rats)) was fed to the laboratory animals. Also, the laboratory animals were adapted to the changed environment 1 week prior to experimentation.

TABLE 3

| | Grafted Materials |
|---|---|
| Negative control | None |
| Positive control | MBCP (HA 60% + β-TCP 40%) |
| Comparative group | Si-substituted HA 100% |
| Experimental group A | Si-substituted HA 70.4% + β-TCP 29.6% |
| Experimental group B | Si-substituted HA 59.2% + β-TCP 40.8% |
| Experimental group C | Si-substituted HA 50.1% + β-TCP 49.9% |

2. Grafting Operation

Zoletil 50 (Virbac lab., France) and Rompun (Bayer, Korea) were mixed in a ratio of 6:4, and 1 ml/kg of the resulting mixture was intramuscularly injected (IM) into each of the rats of the negative control, the positive control, the comparative group and the experimental groups A to C. Rats' hair was cut on the center of rat heads, and the hair-cut areas were sterilized with povidone iodine. A surgical site was marked, and the scalp and the periosteum were cut along a median line from the front of the frontal bone to the crown of the head, then to ⅔ of the parietal bone, thus exposing the top of the skull. A bone defect region was formed by spraying saline on the exposed skull at a rotary speed of 300 to 500 rpm using a trephine bur with a diameter of 8 mm. Then, one corresponding bone graft material per population was grafted into bone defect regions of the rats in each group, and the bone defect regions of the rats were repaired with an absorbent 4/0 vicryl suture. The repaired surgical sites were sterilized with betadine, and all of the subsequent procedures were performed under conventional aseptic conditions. The day after the surgical operation, behavior of the rats was observed, and the surgical sites were sterilized with povidone iodine once more.

3. Sacrifice

The rats were sacrificed 4 and 8 weeks after the grafting of the bone graft material. In order to effectively fix tissues, perfusion fixation was performed at the time of the sacrificing. An equivalent dose of an anesthetic solution used in the surgical operation was administered to anesthetize the rats and cut the rats' pleural cavities. Then, the right lobes of the rats' exposed lungs were incised, and the surgical sites were also spontaneously fixed by continuously administering a 4% paraformaldehyde solution into the rats' left lobes so that a fixing solution could be systemically circulated through the rat vessels. Subsequently, the grafted bone sites and their peripheral sites was incised and immersed in a 4% paraformaldehyde solution. The tissues immersed in a fixing solution were kept at room temperature while being shaked.

4. Collection of Specimens

The tissues were fixed in a 4% paraformaldehyde solution for 2 weeks. Then, the fixed tissues were washed with PBS, and then demineralized for 2 weeks in a Morse's demineralization solution (10% sodium citrate tribasic dehydrate (Sigma chemical co., No.S4641, MO, USA)+25% formic acid (Sigma chemical co., No.F0507, MO, USA)). Subsequently, the demineralized tissues were washed three times with PBS (each wash was performed for 30 minutes). Then, the washed tissues were dehydrated with 70 to 100% ethyl alcohol and xylene, and embedded in paraffin. An embedded tissue block was cut to a thickness of 7 μm using a Microtome-Rotary (Leica RM2165, Germany). Each of the cut tissue specimens was put on a slide glass, and then stained with hematoxylin/eosin and Masson's trichrome.

5. Radiological Inspection

The fixed tissues were subjected to X-ray imaging at 70 kVp for 0.1 second. In this case, a distance between an X-ray tube and the tissues was set to 10 cm. The photographed X-ray film was developed, and bone defect regions were then observed on the developed X-ray image.

Tissue 4 weeks after the grafting of the porous composite according to one exemplary embodiment of the present invention into a bone defect region was photographed under an X-ray imaging system and shown in FIGS. 4, and tissue 8 weeks after the grafting of the porous composite according to one exemplary embodiment of the present invention into a bone defect region was photographed under an X-ray imaging system and is shown in FIG. 5.

As shown in FIGS. 4 and 5, it was revealed that the materials grafted into the bone defect regions stayed fixed there.

6. Morphological Inspection

The bone defect regions in the tissue specimens were observed under a microscope. In particular, new bone formation, the presence of inflammation, and the formation of bone tissue around the bone graft material were observed. When the bone tissues were stained with an H&E stain, the nuclei in the demineralized bone tissues were stained violet, but the other tissues were stained pink. In this case, the connective tissues and the newly formed bone tissues were discriminated on the basis of the morphological analyses and staining density. In the case of the Masson's trichrome staining, demineralized bones in the demineralized tissues were stained blue, and the osteoids were stained red.

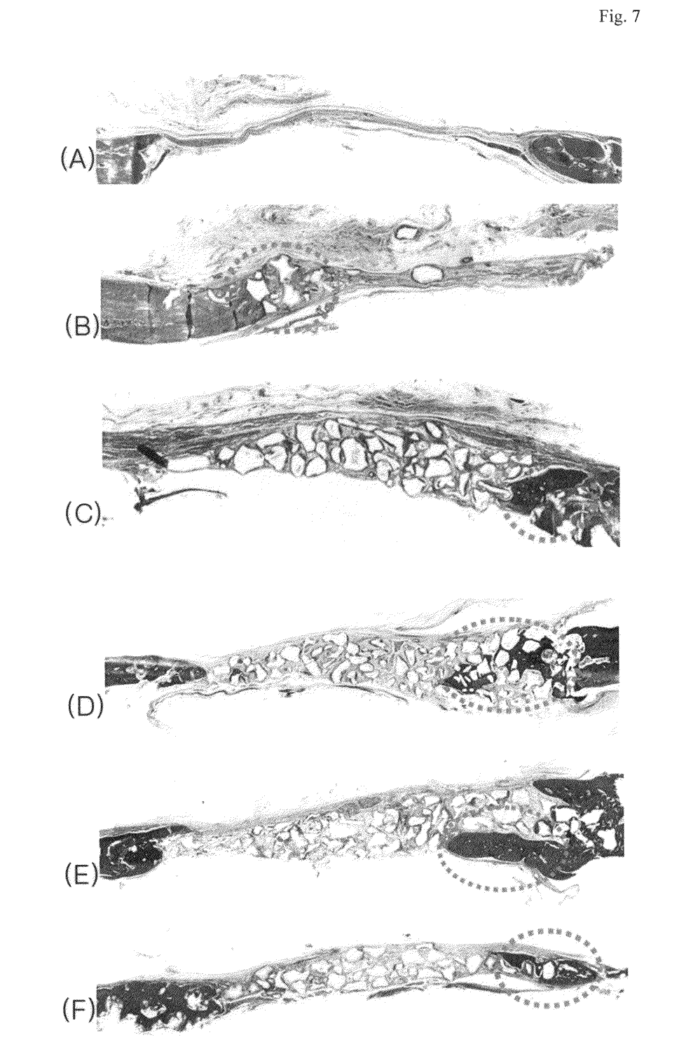

The bone defect region 4 weeks after the grafting of the porous composite according to one exemplary embodiment of the present invention into a bone defect region was observed under a microscope and is shown in FIG. 6, and the bone defect region 8 weeks after the grafting of the porous composite according to one exemplary embodiment of the present invention into a bone defect region was observed under a microscope and is shown in FIG. 7.

As shown in FIGS. 6 and 7, it was revealed that the central region is the bone defect region, and, when bone tissue is stained with a Masson's Trichrome stain, a newly formed region (dark blue) and a region to be newly formed (light blue) are discriminated in the bone tissue whose edge is stained with blue in the bone defect regions. As shown in FIGS. 6C, D, E, F and G, and FIGS. 7B, C, D, E and F, it was confirmed that new bone was formed at the edge of the bone defect region and around the bone graft material, and there was no inflammation in any of the bone tissue. As shown in FIGS. 6A and B, it was revealed that epithelial cells are present but the new bone formation is not observed in the case of the bone graft material-free negative control. Also, it was seen that a void space is formed in the grafted region due to the movement of the bone graft material, as shown in FIG. 6C (positive control), and the regions marked inside circles in FIGS. 6D, E, F and G and FIGS. 7C, D, E and F are regions where new bone began to be formed and was somewhat mineralized from the edge of the bone defect regions.

7. Measurement of New Bone Mineral Density

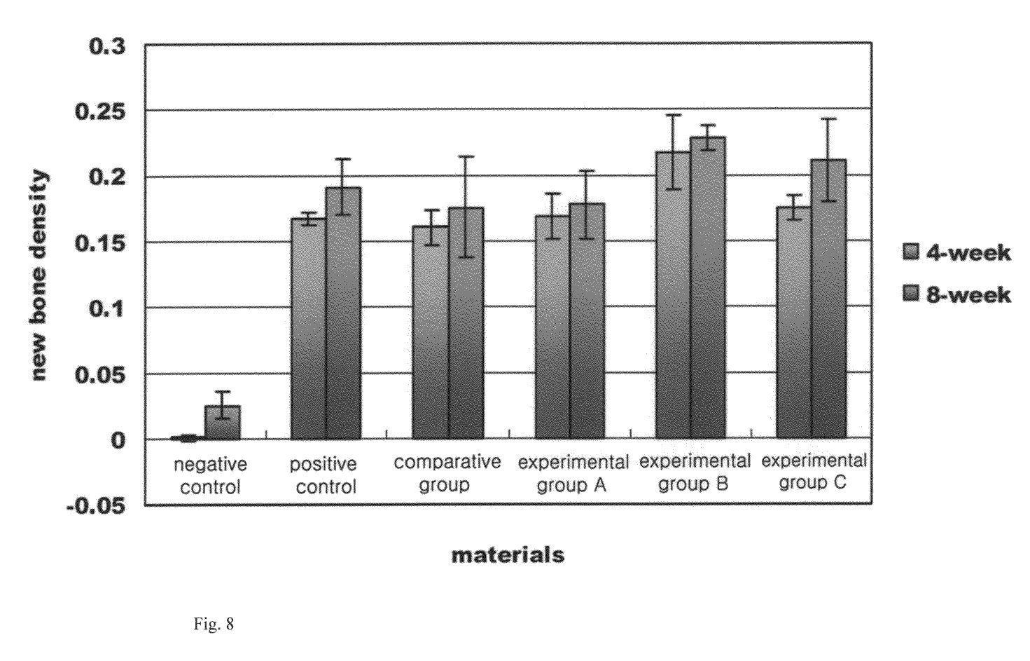

In order to measure the amount of newly formed bone, a bone defect region was referred to as a bone augment area, and newly formed bone was referred to as a new bone area. Then, an area ratio of the new bone area to the bone augment area was measured using an image analyzer. The results are shown in FIG. 8. The new bone mineral density was calculated according to the following Equation 1.

New Bone Mineral Density=[New Bone Area/Bone Augment Area] Equation 1

As shown in FIG. 8, it was revealed that the newly formed bone had a very low bone mineral density 4 weeks and 8 weeks after the grafting of the porous composite according to the present invention into the bone defect region in the case of the negative control (None). On the contrary, it was confirmed that new bone formation is normally seen in all the positive control (MBCP), the comparative group and the experimental groups (A, B and C). Among these, the experimental group C (Si-HA:β-TCP=approximately 60:40) shows the best new bone formation, and its bone formation is more rapid than that of the positive control.

### INDUSTRIAL APPLICABILITY

As described above, the porous composite according to one exemplary embodiment of the present invention may be prepared by subjecting natural coral to hydrothermal and solvothermal reactions to prepare silicon-substituted hydroxyapatite (Si-HA) and subjecting the Si-HA to a heat treatment process. Therefore, the Si-HA and β-TCP may be mixed in the porous composite. As a result, the porous composite according to one exemplary embodiment of the present invention is excellent in biocompatibility and biodegradability and is biocompatible. Also, the porous composite according to one exemplary embodiment of the present invention functions to maintain the microstructure of coral and is similar to natural bone in terms of composition and shape. Accordingly, the porous composite according to one exemplary embodiment of the present invention may be effectively used as a bone tissue repairing material and a bone graft material that can substitute for human hard tissue.

What is claimed is:

1. A porous composite, comprising silicon-substituted hydroxyapatite and β-tricalcium phosphate (β-TCP), with silicon-substituted hydroxyapatite to β-TCP weight ratio of 50˜90%:10˜50%.

2. The porous composite according to claim 1, wherein a content of the substituted silicon in the porous composite is in a range of 0.1 to 2.0% by weight, based on total weight of the porous composite.

3. A method for preparing the porous composite defined in claim 1, the method comprising:
1) preparing silicon-substituted hydroxyapatite by subjecting natural coral to hydrothermal reaction in a (NH₄)₂HPO₄ solution, followed by subjecting the hydrothermally reacted coral to solvothermal reaction in a silicon acetate/acetone-saturated solution; and
2) converting a portion of the silicon-substituted hydroxyapatite into β-tricalcium phosphate (β-TCP) by performing a heat treatment process at 800˜1200° C. for 1˜6 hours on the silicon-substituted hydroxyapatite prepared in Step 1.

4. The method according to claim 3, wherein the hydrothermal reaction of Step 1 is performed at 200° C. for 16˜20 hours.

5. The method according to claim 3, wherein the solvothermal reaction of Step 1 is performed at 50˜90° C. for 30˜50 hours.

6. A bone tissue repairing material, comprising the porous composite defined in claim 1.

7. A bone graft material, comprising the porous composite defined in claim 1.
